SEQUENCE LISTING (8 sequences)

SEQ ID NO: 1 - DNA, 22 bp, Artificial Sequence
  Laboratory synthesis
gcacagaatccttggtgaacag

SEQ ID NO: 2 - DNA, 22 bp, Artificial Sequence
  Laboratory synthesis
atggaaggtgaatgtttcagca

SEQ ID NO: 3 - DNA, 22 bp, Artificial Sequence
  Laboratory synthesis
tgagcaagtttgaaacgcacat

SEQ ID NO: 4 - DNA, 21 bp, Artificial Sequence
  Laboratory synthesis
agctcttctcctgccgtctct

SEQ ID NO: 5 - DNA, 22 bp, Artificial Sequence
  Laboratory synthesis
gaagcagaagaggatcaccttg

SEQ ID NO: 6 - DNA, 21 bp, Artificial Sequence
  Laboratory synthesis
ttcttaaggctgagctgcaag

SEQ ID NO: 7 - DNA, 20 bp, Artificial Sequence
  Laboratory synthesis
cctcagcctccagcagatgc

SEQ ID NO: 8 - DNA, 21 bp, Artificial Sequence
  Laboratory synthesis
ccgcttgcacttcaccctttg

(19) United States
(12) Patent Application Publication
(10) Pub. No.: US 2023/0048690 A1
(43) Pub. Date: Feb. 16, 2023

(54) SCAFFOLD WITH HIERARCHICAL STRUCTURE, PREPARATION METHOD THEREFOR AND APPLICATION THEREOF

(71) Applicant: Tsinghua University, Beijing (CN)

(72) Inventors: Rui YAO, Beijing (CN); Lu FENG, Beijing (CN)

(21) Appl. No.: 17/790,952

(22) PCT Filed: Jul. 7, 2020
(86) PCT No.: PCT/CN2020/100525

§ 371 Date: Jul. 5, 2022

(30) Foreign Application Priority Data
Jan. 6, 2020 (CN) 202010011513.8

(51) Int. Cl. C12N 5/00

(52) U.S. Cl. CPC C12N 5/0062; C12N 2535/00

(57) ABSTRACT

A scaffold with hierarchical structure, a preparation method therefor and an application thereof. The scaffold with hierarchical structure has a structure ranging from centimeters to micrometers, and is used in the fields of three-dimensional cell culture, in vitro large-scale amplification, in vitro tissue-like construction, tissue engineering and regenerative medicine, pathological model research, new drug research and development, drug toxicology research and the like.

### CROSS-REFERENCE TO RELATED APPLICATION

The present disclosure claims priority to Chinese patent application, No. 202010011513.8, filed on Jan. 6, 2020, titled “Scaffold with Hierarchical Structure, Preparation Method Therefor and Application Thereof”, which is hereby incorporated by reference in its entirety.

### TECHNICAL FIELD

The present disclosure relates to the technical field of biological tissue engineering, in particular to a scaffold with hierarchical structure, a preparation method therefor and an application thereof.

### BACKGROUND

In general, a single dose of cell transplantation in an adult must reach 10⁸ to 10⁹ cells to achieve effective functions. Meanwhile, the safety, efficacy, and minimal batch-to-batch differences of stem cell therapy products must be guaranteed to ensure a consistent therapeutic effect. Therefore, it is imperative to develop cost-effective strategies for stem cell amplification, differentiation and/or functional maintenance, and harmless harvest.

Compared with planar culture, three-dimensional (3D) cell culture has great advantages in reducing the variances between in vitro cultures and natural tissues by re-establishing cell-cell and cell-matrix interactions. For the microscale culture system, microcarriers have been used for large-scale amplification platforms of mesenchymal stem cells, embryonic stem cells or induced pluripotent stem cells. For amplification platforms with larger dimensions, 3D scaffolds composed of a natural and/or synthetic biomaterial have been used to expand hematopoietic stem cells, mesenchymal stem cells and embryonic stem cells. Among matrix materials, alginate and gelatin are widely used due to good biocompatibility, biodegradability and mild cross-linking conditions. In addition, Moreover, the hydration characteristic of alginate and reversible cross-linking of gelatin at a cell culture temperature allow noninvasive and nondestructive cell harvesting under physiological conditions. However, challenges still exist in developing a large-scale cell culture system that meets the following criteria simultaneously: 1) large-quantity cell absorption and uniform distribution throughout the whole culture system, (2) sufficient nutrient and mass transfer and adequate mechanical stability in long-term culture, and 3) collection of cells/cell clusters with maintained phenotype and functions.

### SUMMARY

The embodiments of the present disclosure provide a scaffold with hierarchical structure having a high porosity and permeability, high cell load, good mechanical properties and good biological properties, which can be used for three-dimensional (3D) cell culture, and the cells in the scaffold can be recovered nondestructively.

A scaffold with hierarchical structure, including a scaffold body, wherein,
there are big interconnected pores with an average pore diameter of 10 to 500 μm inside the scaffold body;
a porosity of the scaffold body is 10% to 95%; and
a Young's modulus of the scaffold body is 0.1 kPa to 10 MPa.

In some embodiments of the present disclosure, the macro structure of the scaffold body is columnar, blocky, lamellar, cystic or tubular, or a combination of any shapes.

In some embodiments of the present disclosure, the scaffold body is a cylinder, a cube or a prism.

In some embodiments of the present disclosure, the scaffold body has big interconnected pores with an average pore diameter of 80 to 200 μm.

In some embodiments of the present disclosure, the porosity of the scaffold body is 50% to 95%.

In some embodiments of the present disclosure, the porosity of the scaffold body is, for example, 20%, 30%, 40%, 50%, 60%, 70%, 80%, 90% or 95%.

In some embodiments of the present disclosure, the Young's modulus of the scaffold body is 30 to 500 kPa.

In some embodiments of the present disclosure, the Young's modulus of the scaffold body is 0.1 kPa, 0.5 kPa, 1 kPa, 1.5 kPa, 2 kPa, 3 kPa, 4 kPa, 5 kPa, 6 kPa, 7 kPa, 8 kPa, 9 kPa, 10 kPa, 0.1 MPa, 0.5 MPa, 1 MPa, 1.5 MPa, 2 MPa, 3 MPa, 4 MPa, 5 MPa, 6 MPa, 7 MPa, 8 MPa, 9 MPa or 10 MPa.

In some embodiments of the present disclosure, the scaffold body further includes at least one hollow channel. Further, the hollow channel runs through the top and bottom of the scaffold body.

In some embodiments of the present disclosure, the at least one hollow channel is two, three, four or more hollow channels.

In some embodiments of the present disclosure, a diameter of the hollow channel is 0.1 to 5 cm, for example, 2 cm.

In some embodiments of the present disclosure, a ratio of height to diameter (referring to an outside diameter) of the scaffold body is (0.1 to 10) : (10 to 0.1), for example, 1:1.

In some embodiments of the present disclosure, the height of the scaffold body is 0.1 to 8 cm, for example, 6 cm; and/or, the diameter (referring to an outside diameter) of the scaffold body is 0.1 to 8 cm, for example, 6 cm.

In some embodiments of the present disclosure, the porosity of the scaffold body is 75% to 90%.

In some embodiments of the present disclosure, the scaffold body has a three-dimensional structure with an upper size of 1 to 50 cm. In some specific embodiments, the dimension of the three-dimensional structure is 1 cm×1 cm×0.5 cm.

In some embodiments of the present disclosure, the scaffold body is composed of a microfilament material of about 50 to 800 μm. In some embodiments of the present disclosure, the scaffold body includes hollow channels with an interval of 0.1 to 1000 mm.

In some embodiments of the present disclosure, the scaffold with hierarchical structure has good elasticity.

In some embodiments of the present disclosure, when the scaffold with hierarchical structure is compressed, it exhibits at least 20% to 70% or higher compression strain without permanent deformation or mechanical damage.

In some embodiments of the present disclosure, the scaffold body is made of a biocompatible material.

In some embodiments of the present disclosure, the biocompatible material is selected from a natural material and/or an artificial synthetic material.

In some embodiments of the present disclosure, the natural material is at least one selected from alginate, alginate derivatives, gelatin, gelatin derivatives, agar, matrix gel, collagen, collagen derivatives, hyaluronic acid, hyaluronic acid derivatives, cellulose, cellulose derivatives, proteoglycan, proteoglycan derivatives, glycoprotein, glycoprotein derivatives, chitosan, chitosan derivatives, laminin, fibronectin and fibrin, silk fibroin, silk fibroin derivatives, vitronectin, osteopontin, peptide hydrogel and DNA hydrogel, and preferably the natural material is sodium alginate and/or gelatin.

In some embodiments of the present disclosure, the synthetic material is at least one selected from polyglycolic acid, polylactic acid, polylactic acid-glycolic acid copolymer, polyglutamic acid-polyethylene glycol, polycaprolactone, polytrimethylene carbonate, polyglycolic acid, polyethylene glycol-polydioxanone, polyethylene glycol, polytetrafluoroethylene, polyoxyethylene, polyethylene vinyl acetate, polytrimethylene carbonate, poly(p-dioxanone), polyether ether ketone, and derivatives and polymers thereof, and preferably the synthetic material is polyglycolic acid or polylactic acid.

In some embodiments of the present disclosure, the crosslinking agent used for preparing the scaffold body is at least one selected from divalent cations, genipin, glutaraldehyde, adopyl diacidhydrizine, epichlorohydrin, carbodiimide, thrombin and derivatives thereof, and preferably the crosslinking agent is calcium chloride.

In some embodiments of the present disclosure, the scaffold body is made of polyglycolic acid and fibrin, and the crosslinking agent is thrombin.

The scaffold with hierarchical structure of the present disclosure is controllable in structure, and has controllable hierarchical structure from centimeter scale to micron scale, and the macro structure can be customized and the micro pores can be adjusted.

The scaffold with hierarchical structure prepared according to the present disclosure is more suitable for the culture of stem cells, such as liver stem cells (Life Technologies) and embryonic stem cells (ATCC) and the like.

The present disclosure also provides a preparation method for the above scaffold with hierarchical structure, which includes the following steps:

1) preparing a precursor solution with a biocompatible material and a corresponding crosslinking agent;

2) preparing a three-dimensional structure body using the precursor solution as raw material;

3) freezing the three-dimensional structure body; and

4) drying the frozen three-dimensional structure body to obtain the scaffold with hierarchical structure.

It is found that by preparing a three-dimensional structure body from the precursor solution followed by freezing and drying, not only macropores are formed, but also a scaffold with hierarchical structure is formed.

According to the preparation method for the scaffold with hierarchical structure of the present disclosure, the biocompatible material having the same meaning as above is selected from a natural material and/or a synthetic material, and is mainly some hydrogel materials with biocompatibility.

In some specific embodiments of the present disclosure, a mass percentage concentration of the biocompatible material is 0.1% to 80%, and preferably 1% to 25%.

According to the preparation method for the scaffold with hierarchical structure of the present disclosure, the crosslinking agent is one or more substances selected from divalent cations represented by calcium chloride, genipin, glutaraldehyde, adopyl diacidhydrizine, epichlorohydrin, carbodiimide, thrombin and their derivatives. In some specific embodiments of the present disclosure, the crosslinking agent is calcium chloride.

In some specific embodiments of the present disclosure, the mass percentage concentration of a crosslinking solution is 0.1 mM to 10 M, and preferably 1 mM to 100 mM.

In some specific embodiments of the present disclosure, the biocompatible material and the crosslinking agent solution are mixed at a volume ratio of 1000:1 to 1:1000, and preferably 10:1 to 1:10.

In some specific embodiments of the present disclosure, the biocompatible material is prepared into a solution (preferably using a sodium chloride solution as a solvent), which is then prepared into a precursor solution with the crosslinking agent solution.

In some specific embodiments of the present disclosure, the biocompatible material is alginate and gelatin, and the crosslinking solution is calcium chloride.

Alginate and gelatin are natural biomaterials with good cytocompatibility. Alginate can be pre-crosslinked rapidly after being mixed with calcium ions and can be degraded under physiological conditions. Gelatin is temperature-sensitive, and reversible crosslinking of gelatin can be realized by adjusting temperature. A scaffold with hierarchical structure can be prepared with a precursor solution containing alginate, gelatin and calcium chloride.

In some specific embodiments of the present disclosure, the scaffold with hierarchical structure is prepared with a precursor solution which is prepared by evenly mixing a polyglycolic acid solution with a concentration of 1% to 25% (preferably using a sodium chloride solution with a concentration of 0.1% to 10% as a solvent), a fibrinogen solution with a concentration of 1% to 25% (preferably using a sodium chloride solution with a concentration of 0.1% to 10% as a solvent) and a thrombin solution with a concentration of 1 to 2000 mM, and the scaffold has the advantages of good cell compatibility, high porosity for cell seeding and growth, suitable pore size for cell growth, similar mechanical properties to natural tissues, and non-destructive collection of cells.

In some specific embodiments of the present disclosure, in the precursor solution, the concentration of polyglycolic acid is 0.1% to 21%, the concentration of fibrinis 0.1% to 21%, and the concentration of thrombin is 0.1 to 1000 mM.

According to the preparation method for the scaffold with hierarchical structure of the present disclosure, the three-dimensional structure body can be prepared with the above precursor solution according to a pre-designed structure by the following method: casting mold method (or process), lost foam mold method (or process), biological 3D printing method (or process), inkjet printing method (or process), fused deposition molding method (or process), electrostatic spinning method (or process), electrostatic driving printing method (or process), particle leaching method (or process), gas foaming technology (or process), stereo lithography technology (or process), laser sintering technology (or process).

In some specific embodiments of the present disclosure, the casting mold method (or process) is used.

In some specific embodiments of the present disclosure, the lost foam mold method (or process) is used.

In some specific embodiments of the present disclosure, the biological 3D printing method (or process) is used.

According to the preparation method for the scaffold with hierarchical structure of the present disclosure, in step (3), a solid three-dimensional structure is obtained by freezing the three-dimensional structure body. Wherein, the three-dimensional structure body is subjected to a stepwise freezing and preferably incubated at 4° C. for 0.5 to 24 h, then at −20° C. for 0.5 to 48 h, and then at −80° C. for 0.5 to 48 h. By this method, big interconnected pores can be obtained, which are suitable for cell seeding and long-term culture and can improve the mechanical properties of the scaffold, rendering it suitable for operation and transportation.

According to the preparation method for the scaffold with hierarchical structure of the present disclosure, in step (4), the scaffold with hierarchical structure is obtained by drying the frozen three-dimensional structure. Wherein, the frozen three-dimensional structure body is dried by vacuum freeze drying, and preferably under a condition of −4° C. to −80° C. and 1 to 1000 Pa.

According to the preparation method for the scaffold with hierarchical structure of the present disclosure, the macro dimension of the scaffold with hierarchical structure can be adjusted, for example, by means of the dimension and structure of the cavity inside the mold and computer modeling. The scaffold with hierarchical structure also be made into a blocky, lamellar, cystic or tubular form or a combination of any shape as needed.

The present disclosure also provides a scaffold with hierarchical structure prepared by the above-mentioned preparation method.

The present disclosure also provides at least one application of the scaffold with hierarchical structure in the following aspects: 1) in vitro cell culture and/or large-scale amplification; 2) drug development, drug screening, drug detection or drug test; 3) construction of a pharmacological model, a pathological model and a tissue/organ model; 4) preparation of materials for tissue repair or regeneration in vivo; and 5) preparation of orthopedic or plastic implants.

The present disclosure also provides a three-dimensional cell culture method, including inoculating cells or a mixture of cells and a biocompatible material into the mentioned scaffold with hierarchical structure for three-dimensional culture. Further, the method also includes a step of cell collection and/or detection.

Wherein, the cells are selected from one or more of the following cells: embryonic stem cells from various sources, pluripotent stem cells, induced pluripotent stem cells, stem cells from various organs, progenitor cells from various organs, mesenchymal stem cells, cells differentiated from various stem cells by induced differentiation, fibroblasts from various organs, epithelial cells from various organs, epidermal cells from various organs, endothelial cells from various organs, muscle cells from various organs, amniotic cells, cone cells, nerve cells, blood cells, red blood cells, white blood cells, platelets, vascular cells, phagocytes, immune cells, lymphocytes, eosinophils, basophils, plasma cells, mast cells, antigen presenting cells, cells of mononuclear phagocyte system, melanocytes, chondrocytes, bone-derived cells, smooth muscle cells, skeletal muscle cells, cardiac muscle cells, secretory cells, adipocytes, ciliated cells, pancreatic cells, renal cells, intestinal mucosa cells, hepatocytes, stem cells or progenitor cells from liver, hepatic macrophages, kupffer cells, astrocytes, biliary epithelial cells, sinusoidal endothelial cells and cells from other tissues and organs, and various tumor cells, various cells for immunotherapy, various cells and cell lines obtained after gene editing, and virus packaging or modification.

Based on the mentioned reasons, further preferably, the cells are stem cells, and more preferably embryonic stem cells or liver stem cells.

Where, the biocompatible material is at least one material of alginate, alginate derivatives, gelatin, gelatin derivatives, agar, matrix gel, collagen, collagen derivatives, hyaluronic acid, hyaluronic acid derivatives, cellulose, cellulose derivatives, proteoglycan, proteoglycan derivatives, glycoprotein, glycoprotein derivatives, chitosan, chitosan derivatives, laminin, fibronectin and fibrin, silk fibroin, silk fibroin derivatives, vitronectin, osteopontin, peptide hydrogel, and DNA hydrogel, and preferably is collagen and derivatives thereof

Specifically, the cell-loaded scaffold with hierarchical structure prepared by the mentioned method can use a static or dynamic culture system, such as by means of various forms of bioreactor, pulsating culture, chip, and perfusion culture systems.

Specifically, according to the characteristics of the selected biological material, the mentioned method realizes the collection of cells/cell clusters in the scaffold with hierarchical structural under physiological conditions, and the process of collecting cells from the scaffold with hierarchical structure has no effect on the morphology, phenotype and function of cells/cell clusters. The harvested cells/cell clusters can be used for cell biology research, tissue repair, cell transplantation therapy, new drug research and development, drug screening, drug detection, construction of a pathological/pharmacological model and various tissue chip models.

The cell-loaded scaffold with hierarchical structure prepared by the mentioned method is applied in in vitro studies, including but not limited to cell culture, cell amplification, cell biology studies, drug development, drug screening, drug detection, drug test, construction of a pathological model, a pharmacological model and a tissue/organ model, tissue repair or regeneration, and orthopedic or plastic implants.

The present disclosure also includes the three-dimensional cell culture obtained by the above method.

### BENEFICIAL EFFECTS

According to the mentioned technical solution, the present disclosure has at least the following advantages and beneficial effects:

The scaffold with hierarchical structure of the present disclosure can be individualized and customizable, has high cell load, high porosity and permeability, adjustable pore size and high elasticity modulus, and allows transplantation through injection.

1) The scaffold with hierarchical structure in the present disclosure has a high cell loading rate. Due to the hierarchical structure and big pores of the scaffold with hierarchical structure, cells can be evenly distributed in the scaffold and a high loading rate may be achieved, and the scaffold can load drugs and/or cells as a drug carrier and/or a therapeutic implant.

2) The scaffold with hierarchical structure in the present disclosure has a good biocompatibility. The scaffold with hierarchical structure in the present disclosure adopts a biocompatible material as a matrix material, it has a very good biocompatibility and can be used for in vivo implantation.

3) The scaffold with hierarchical structure in the present disclosure has good mechanical properties. The scaffold with hierarchical structure in the present disclosure has higher mechanical stability over a conventional gel scaffold / a gel scaffold with the same composition.

4) The scaffold with hierarchical structure in the present disclosure has a good biological performance. The scaffold with hierarchical structure of the present disclosure can load a variety of cells and significantly promote cell proliferation, cell aggregation, cell activity, and maintain and improve cell functions.

5) The scaffold with hierarchical structure in the present disclosure can realize nondestructive collection of cells. The scaffold with hierarchical structure in the present disclosure uses a biocompatible material as a matrix material, and it can be hydrolyzed under physiological conditions to realize non-destructive collection of cells/cell clusters in the scaffold.

### BRIEF DESCRIPTION OF DRAWINGS

FIG. 1 is a diagram of a scaffold with hierarchical structure according to an embodiment of the present disclosure.

FIG. 2 is a diagram of scaffolds with hierarchical structure according to some embodiments of the present disclosure.

FIG. 3 is a diagram showing liver stem cells cultured in the scaffold with hierarchical structure according to Example 1 of the present disclosure. FIG. 3A shows the distribution and clustering of liver stem cells after proliferation in the scaffold for 7 days. FIG. 3B shows transcription levels of liver specific genes in liver stem cells in planar cultures (2D), in 3D scaffolds with hierarchical structure and harvested after hydration of 3D scaffolds, under the same condition.

FIG. 4 is a schematic diagram of a grid-like structure used in Example 2 of the present disclosure and prepared by single nozzle 3D printing.

FIG. 5 shows morphology of scaffolds with hierarchical structure prepared by three- dimensional printing in Example 2 of the present disclosure. FIG. 5A is a schematic diagram of the grid-like 3D structure body formed by 3D printing; FIG. 5B is a top view of the scaffold with hierarchical structure prepared by 3D printing technology; FIG. 5C is a side view of the scaffold with hierarchical structure prepared by 3D printing technology; and FIG. 5D shows a micromorphology of the scaffold with hierarchical structure observed by SEM.

FIG. 6 is a diagram showing embryonic stem cells cultured in the scaffold with hierarchical structure according to Example 2 of the present disclosure. FIG. 6A shows distribution and clustering of embryonic stem cells after 4 days of culture in the scaffold with hierarchical structure under a light microscope; FIG. 6B shows the proliferation of embryonic stem cells in planar cultures (2D) and 3D scaffolds with hierarchical structure after 4 days of culture relative to that at the 0 day; and FIG. 6C shows transcription levels of totipotent genes of liver stem cells in planar cultures (2D), in 3D scaffolds and harvested after hydration of 3D scaffolds, under the same condition.

### DETAILED DESCRIPTION OF THE EMBODIMENTS

The following embodiments are used to illustrate the present disclosure but are not used to limit the scope of the present disclosure. If not specified in particular, the technical means used in the Examples are general means known to people skilled in the art, and the raw material used is commercial commodity.

The percent sign “%” involved in the present disclosure, if not specified in particular, refers to a mass percentage; but for the percentage involved in a solution, it refers to solute grams of the solute in 100 mL solution unless otherwise specified.

Unless otherwise defined, all technical terms used in the present disclosure have the same meaning as those people skilled in the art would understand.

The term “crosslinking solution” used in the present disclosure refers to the solution that plays the function of crosslinking a material having biocompatibility in the preparation of the precursor solution, which can be a material known by people skilled in the art that can crosslink the material having biocompatibility to form a solution with a certain viscosity, such as a calcium chloride solution, preferably 1 to 100 mM, for example, a calcium chloride solution with a concentration of 5 mM.

The term “three-dimensional printing” used in the present disclosure refers to the three-dimensional precise deposition using a raw material compatible with three-dimensional printing through a method matched to an automatic or semi-automatic and computer-aided three-dimensional molding device (such as a three-dimensional printer).

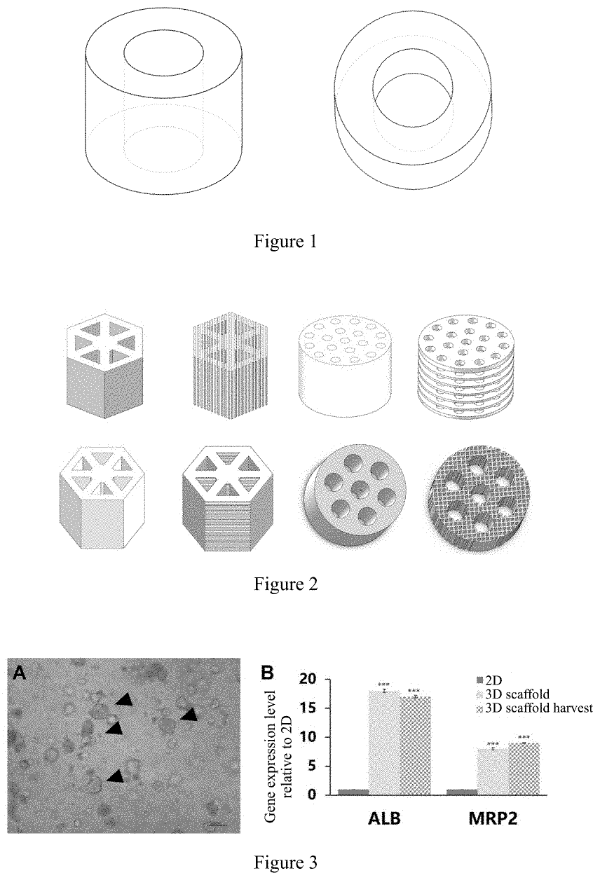

FIGS. 1 to 2 are two diagrams of scaffolds with hierarchical structure according to Examples of the present disclosure.

### Example 1: Preparing a Scaffold with Hierarchical Structure by Casting Mold Method

The present Example provides a scaffold with hierarchical structure, as shown in FIG. 1, including a scaffold body, wherein, there are big interconnected pores with an average pore diameter of about 100 μm inside the scaffold body, the porosity of the scaffold body is 75%, and the Young's modulus of the scaffold body is 220 kPa. The height of the scaffold body is 6 cm, and the diameter (refer to the outside diameter) of the scaffold body is 6 cm. The scaffold body further includes a hollow channel, and the diameter of the channel is 2 cm.

The preparation method for the above scaffold with hierarchical structure provided by the present Example, includes the following steps.

1. Preparing a biomaterials solution

21% polyglycolic acid solution: mixing polyglycolic acid powder (Sigma-Aldrich) with 0.9% sodium chloride solution at a mass ratio of 21:100, stirring the mixture with a magnetic stirrer for about 5 min and meanwhile heating the mixture at 100° C. until the polyglycolic acid powder is uniformly dissolved, and after cooling, sub-packing the mixture and preserving the resultant at 4° C.

21% fibrin solution: mixing fibrinogen powder (Sigma-Aldrich) with 0.9% sodium chloride solution at a mass ratio of 21:100, and heating the mixture at 37° C. until the fibrinogen powder is uniformly dissolved.

2. Preparing a crosslinking solution

600 mM thrombin solution: dissolving thrombin powder in deionized water to prepare 600 mM thrombin solution as the crosslinking solution.

3. Preparing a precursor solution

Mixing the above prepared 21% polyglycolic acid solution, 21% fibrinogen solution and 600 mM thrombin solution uniformly, to obtain the precursor solution with a concentration of 7% polyglycolic acid, 7% fibrinogen and 200 mM thrombin.

4. Preparing a three-dimensional structure body by casting mold method

Pouring the above precursor solution into a preset mold, as shown in FIG. 1, to form a three-dimensional structure body with a hollow cylinder shape, and the three-dimensional structure has an outer diameter of 6 cm, a hollow diameter of 2 cm and a height of 6 cm.

5. Freezing the prepared three-dimensional structure body

Freezing the pregel three-dimensional structure body stepwise. Specifically, the steps are preserving the three-dimensional structure body at 4° C. for 24 h, and then at −20° C. for 48 h.

6. Drying the frozen three-dimensional structure body

Drying the three-dimensional structure body under a condition with a low temperature of −80° C. and a high vacuum of 500 Pa for 24h to form a scaffold with hierarchical structure. Sterilizing the scaffold by UV irradiation for 2 h and preserving it under a sterile condition for subsequent biological application of the scaffold.

Culturing liver stem cells using the scaffold with hierarchical structure prepared in Example 1, and the specific method is as follows.

7. Inoculating liver stem cells into the scaffold with hierarchical structure

Dispersing liver stem cells (Life Technologies) uniformly in their cell culture medium at a density of 10⁴ cells/mL to form a cell suspension. Adding 1 mL of the cell suspension into the three-dimensional cell scaffold dropwise, and keeping it in a cell incubator for 24 h.

8. Detecting distribution, proliferation, clustering and metabolic activity of cells in the scaffold

Providing a sufficient amount of cell culture medium for the scaffold after inoculation of cells, culturing the cells under a conventional cell culture condition (in an incubator, 37° C., 5% CO₂), and replacing fresh medium every 2 to 3 days.

FIG. 3 is a diagram showing liver stem cells cultured in the scaffold with hierarchical structure prepared by casting mold method according to Example 1.

FIG. 3A shows morphology of liver stem cells cultured in the scaffold with hierarchical structure after 7 days. Under a light microscope, it can be seen that the cells were uniformly distributed in the scaffold and formed uniform clusters as shown by the arrows in the figure.

Detecting cells in the three-dimensional structure body by live-dead staining at day 0 and day 7. In the present disclosure, a mixed solution of 2 uM Calcein-AM (Dojindo, C326) and 4.5 uM PI (Dojindo, P346) were used to stain live cells (green color) and dead cells (red color) respectively, and the staining was performed in dark for 15 minutes. Recording and observation were performed with laser scanning confocal microscopy (Laser Scanning Confocal Microscope, LSCM) (Nikon, Z2). After completion of the printing, the survival rate of the cells in the structure was about 98% at day 0.

Detecting the proliferation of liver stem cells in the scaffold with hierarchical structure on day 3 and day 7 respectively. Under the same initial cell load, culture environment, culture medium and culture conditions, there is no significant difference between the metabolic activity of the liver cells cultured in the scaffold with hierarchical structure prepared by the present disclosure and that in two-dimensional cultures at every detection time point, and the metabolic activity was detected by using the commonly used cell metabolic activity detection kit (CellTiter-Blue® Cell Viability Assay, Promega).

9. Detecting functions of liver stem cells in the scaffold

In order to detect functions of liver stem cells in the scaffold, immunofluorescence staining was used to detect the expression of mature hepatocyte-specific protein makers (such as ALB and MRP2).

Immunofluorescence staining: washing the structure with phosphate buffer (Phosphate Buffer Saline, PBS) (BI, 02-024-1AC); fixing it in 4% paraformaldehyde at room temperature for 30 minutes, and then washing it 3 times with PBS, each for 5 minutes; blocking it in a mixture containing 0.3% Triton-X (Sigma, X100) and 5% bovine serum albumin (Bovine Serum Albumin, BSA) (Multicell, 800-096-EG) for 1 hour; sucking out the blocking buffer, adding the diluted primary antibody (containing 0.3% Triton-X and 1% BSA), ALB (Abcam, ab83465) and MRP2 (Abcam, ab3373), and incubating at 4° C. overnight. Washing it 3 times with PBS, each for 5 minutes; adding corresponding second antibody, Alexa Fluor® 594 (Abcam, ab150080) and Alexa Fluor® 488 (Abcam, ab150113), incubating it at room temperature in dark for 2 hours, and then washing it 3 times with PBS, each for 5 minutes; and then adding DAPI to stain cell nucleus and incubating it at room temperature in dark for 5 minutes. Observation and recording were performed with laser scanning confocal microscope (Laser Scanning Confocal Microscope, LSCM) (Nikon, Z2).

10. Non-destructive collecting of cell clusters in the scaffold and maintenance of phenotype and function of the harvested cell clusters

The scaffold with hierarchical structure in the present disclosure is composed of a hydrolyzable natural material, which can be hydrolyzed under physiological conditions so that the non-destructive collection of cells in the scaffold is achieved.

By qPCR technology, detecting the transcription level of genes related to mature hepatocytes in cell clusters in planar cultures, scaffold cultures and harvested after hydrolyzing the scaffold. Results are shown in FIG. 3B, the gene transcription level of cells in 3D scaffold was significantly higher than that in planar culture. The expression level of ALB of cells in 3D scaffold was 15 times higher than that in planar culture, and the expression level of MRP2 of cells in 3D scaffold was 4 times higher than that in planar culture. This indicates that after culturing in the scaffold for 7 days, liver stem cells were significantly differentiated into mature hepatocytes. Meanwhile, there is no difference between the gene expression levels of ALB and MRP2 of the cell clusters harvested after hydration and those of the cells in the 3D scaffold, indicating that the process of obtaining cells by hydrolyzing the scaffold has no effect on the morphology, phenotype and functions of cells.

qPCR Technology

Operation steps for extraction of RNA from cells: washing the structure with PBS once, adding 1 ml Trizol (Gibco, 15596026) into each structure, pipetting up and down to mix uniformly, keeping the resultant at room temperature for 10 minutes, then transferring the mixture to 1.5 ml EP tube, adding 200 μl chloroform, shaking the tube rapidly for 30 seconds, and after keeping the mixture at room temperature for 5 minutes, centrifuging at 4° C. and 12000 g for 10 minutes. Removing supernatant, adding isopropyl alcohol of a same volume into the remaining solution, and centrifuging the resultant at 4° C. and 12000 g for 10 minutes. Removing the supernatant, and washing the pellet with 75% ethyl alcohol. After drying, RNA was obtained, and then dissolved in DEPC water. Concentration and purity of RNA were detected by spectrophotometer (Thermo Scientific).

Operation steps for RNA reverse transcription: using PrimeScript™ II 1st strand cDNA Synthesis Kit (TaKaRa,6210) and operating RNA reverse transcription completely in accordance with the kit instructions. RNA content was adjusted to 5 ng. The primer was Oligo dT Primer. Program for reverse transcription PCR was as follows: 42° C. for 50 min, 95° C. for 5 min, and 4° C. for preservation, and PCR instrument was SimpliAmp™ thermal cycler (ABI).

Operating steps for fluorescence quantitative PCR: using Maxima SYBR Green qPCR Master Mix (Thermo Scientific, K0251) and a kit, operating the fluorescence quantitative PCR completely in accordance with the kit instruction. After adding the reaction solution as required, and placing the reaction plate in a qPCR instrument for detection. The reaction program was as follows: 95° C. for 10 min, [95° C. for 15 s, 60° C. for 30 s]×40 cycles, 72° C. for 30 s, and 72° C. for 10 min. Obtaining the expression of genes at different time points (FIG. 3B).

The primer sequences used for qPCR were as follows (5′-3′):

ALB primer sequences:
Forward:
GCACAGAATCCTTGGTGAACAG
Reverse:
ATGGAAGGTGAATGTTTCAGCA
MRP2 primer sequences:
Forward:
TGAGCAAGTTTGAAACGCACAT
Reverse:
AGCTCTTCTCCTGCCGTCTCT

### Example 2: Preparing a Scaffold with Hierarchical Structure by Single Nozzle Three-Dimensional Printing

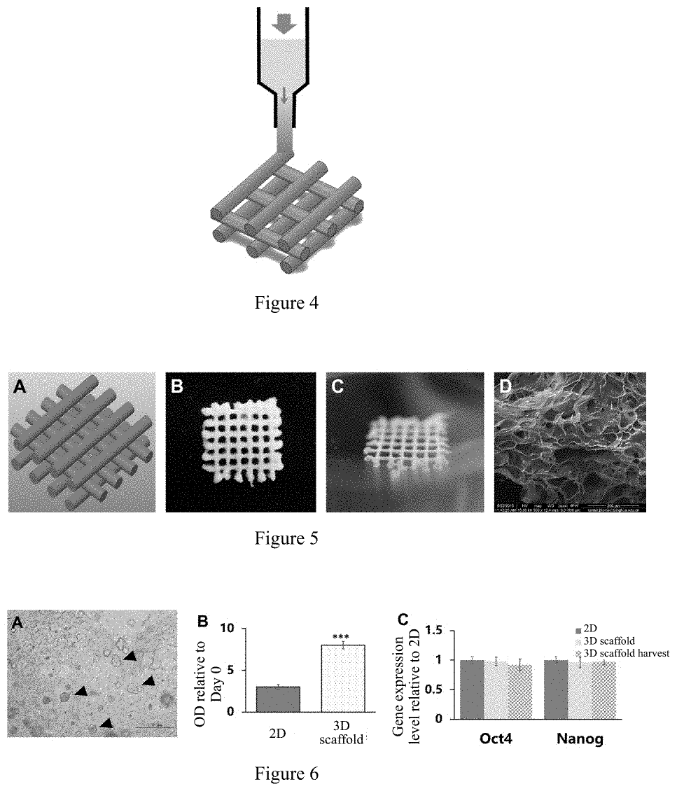

This embodiment provides a scaffold with hierarchical structure, as shown in FIGS. 4 to 5, including a scaffold body, wherein, there are big interconnected pores with an average pore diameter of about 100 μm inside the scaffold body, the porosity of the scaffold body is 95%, and the Young's modulus of the scaffold body is 30 kPa. The scaffold body has a three-dimensional structure of 1 cm×1 cm×0.5 cm in size. The scaffold body is composed of microfilaments of about 300 μm. The scaffold body includes hollow channels with an interval of about 1 mm.

Further, the scaffold body is composed of a hierarchical structure.

The preparation method for the above scaffold with hierarchical structure provided by the present Example, includes the following steps.

1. Preparing a precursor solution with concentration of 7% polyglycolic acid, 7% fibrinogen and 200 mM thrombin according to the same method as in Example 1.

2. Preparing a scaffold with hierarchical structure by single nozzle 3D printing

Preparing the three-dimensional structure by using a single-nozzle extrusion printer, and the single nozzle 3D printer is shown in FIG. 4. Collecting the precursor solution into a sterile syringe, and loading the sterile syringe into the biological three-dimensional printing equipment (Regenovo, Bio-architect X). The printer is equipped with a non-destructive optical coherence tomography (Optical Coherence Tomography, OCT) system, which can realize non- destructive monitoring during the printing process to ensure the quality of the sample and reduce the difference between batches and within a batch. Under a condition with a support speed of 50 mm/s, a contour speed of 50 mm/s, a mesh speed of 50 mm/s and an extrusion speed of 50 μL/s, the printer performed three-dimensional printing on a bottom platform where was sterile and the temperature can be controlled. The temperature of the bottom platform was set to 0° C. and a three-dimensional structure body of hydrogel with a volume of 3 cm/3 cm/1 cm was formed, the schematic diagram of which is shown in FIG. 5A.

3. Freezing the prepared three-dimensional structure body

Freezing the pregel three-dimensional structure body stepwise, the specific steps were preserving the three-dimensional structure body at 4° C. for 24 h, and then at −20° C. overnight.

4. Drying the frozen three-dimensional structure body

Drying the three-dimensional structure body at a condition with a low temperature of −80° C. and a high vacuum of 500 Pa for 24h, to form a scaffold with hierarchical structure. Sterilizing the scaffold by UV irradiation for 2 h and preserving it under a sterile condition for subsequent biological application. The macro structure of the frozen scaffold with hierarchical structure after drying is shown in FIG. 5B (top view) and FIG. 5C (side view). Observation of the microstructure of the big interconnected pores of the scaffold was performed with scanning electron microscopy (Scanning Electron Microscopy, SEM). The diameters of the big interconnected pores in the scaffold were 100 to 300 μm, as shown in FIG. 5D.

Culturing embryonic stem cells using the scaffold with hierarchical structure prepared in the present Example, and the specific method is as follows.

5. Inoculating embryonic stem cells into the scaffold with hierarchical structure

Dispersing embryonic stem cells (Life Technologies) uniformly in their cell culture medium at a density of 10⁴ cells/mL to form a cell suspension, adding 1 mL of cell suspension into the three-dimensional cell scaffold dropwise, and by a dynamic culturing method, rotating the scaffold added with the cell suspension at a speed of 5000 RPM on a horizontal vibrating screen (Beijing Hinsr Technology Co., Ltd., WD-9405F), and keeping it for 12 h under the cell culture condition (in an incubator, 37° C., 5% CO2).

6. Detecting distribution, proliferation, clustering and metabolic activity of cells in the scaffold

Providing a sufficient amount of cell culture medium for the scaffold after inoculation of cells, culturing it under a conventional cell culture condition (in an incubator, 37° C., 5% CO₂), and replacing fresh medium every 2 to 3 days. FIG. 6A shows morphology of embryonic stem cells cultured in the scaffold with hierarchical structure for 7 days, and the arrows point to the embryonic stem cell clusters. Under a light microscope, it can be observed that the cells were uniformly distributed in the scaffolds and formed clusters having uniform size.

Detecting cells in the three-dimensional structure body by live-dead staining on day 0 and day 7. In the present disclosure, a mixed solution of 2 uM Calcein-AM (Dojindo, C326) and 4.5 uM PI (Dojindo, P346) were used to stain live cells (green color) and dead cells (red color) respectively, and the staining was performed in dark for 15 minutes. Recording and observation were performed using laser scanning confocal microscopy (Laser Scanning Confocal Microscope, LSCM) (Nikon, Z2). After completion of the printing, the survival rate of the cells in the structure was about 99% at day 0.

FIG. 6B shows the proliferation of embryonic stem cells in the scaffold with hierarchical structure printed by three-dimensional printing. Under the same initial cell load, culture environment, culture medium and culture conditions, the liver cells cultured in the scaffold with hierarchical structure prepared by the present disclosure have a significant increase in metabolic activity over that in two-dimensional cultures at every detection time point, and the metabolic activity was detected by using the commonly used cell metabolic activity detection kit (CellTiter-Blue® Cell Viability Assay, Promega).

7. Detecting the totipotency of embryonic stem cells in the scaffold

In order to detect the totipotency of embryonic stem cells in the scaffold, the expression of classical protein markers for totipotency (such as OCT4 and Ecad) were detected by immunofluorescence staining.

Immunofluorescence staining: washing the structure with phosphate buffer (Phosphate Buffer Saline, PBS) (BI, 02-024-1AC); fixing the resultant in 4% paraformaldehyde at room temperature for 30 minutes, and then washing it 3 times with PBS, each for 5 minutes; blocking it in a mixture containing 0.3% Triton-X (Sigma, X100) and 5% bovine serum albumin (Bovine Serum Albumin, BSA) (Multicell, 800-096-EG) for 1 hour; sucking out the blocking buffer, adding the diluted primary antibody (containing 0.3% Triton-X and 1% BSA), OCT4 (Abcam, ab19857) and E-cadherin (Abcam, ab231303), and incubating at 4° C. overnight. Washing the resultant 3 times with PBS, each for 5 minutes; adding corresponding second antibody, Alexa Fluor® 594 (Abcam, ab150080) and Alexa Fluor® 488 (Abcam, ab150113), incubating at room temperature in dark for 2 hours, and then washing 3 times with PBS, each for 5 minutes; and then adding DAPI to stain cell nucleus and incubating at room temperature in dark for 5 minutes. Observation and recording were performed with laser scanning confocal microscope (Laser Scanning Confocal Microscope, LSCM) (Nikon, Z2).

8. Non-destructive collecting of cell clusters in the scaffold and maintenance of phenotype and functions of the harvested cell clusters

The scaffold with hierarchical structure in the present disclosure is composed of a hydrolyzable natural material, and it can be hydrolyzed under physiological conditions so that the non-destructive collection of cells in the scaffold is achieved.

By qPCR technology, detecting the transcription level of classical totipotentcy-related genes in planar cultures, clusters in scaffold and clusters harvested after hydrolyzing the scaffold. Results are shown in FIG. 6C, there was no significant difference in the transcription level of totipotency genes of cells between planar cultures, scaffold cultures and cell clusters harvested after hydrolyzing the scaffold, indicating that the process of culturing cells in the scaffold with hierarchical structure, and hydrolyzing the scaffold to harvest the cells has no effect on the morphology, phenotype and totipotency of cells.

qPCR technology: Operation steps for extraction of RNA from cells: washing the structure with PBS once, adding 1 ml Trizol (Gibco, 15596026) into each structure, pipetting up and down to mix uniformly, keeping the resultant at room temperature for 10 minutes, then transferring the mixture to 1.5 ml EP tube, adding 200 μl chloroform, shaking the tube rapidly for 30 seconds, and after keeping it at room temperature for 5 minutes, centrifuging at 4° C. and 12000 g for 10 minutes. Removing supernatant, adding isopropyl alcohol of a same volume into the remaining solution, and centrifuging the resultant at 4° C. and 12000 g for 10 minutes. Removing the supernatant, and washing the pellet with 75% absolute ethyl alcohol. After drying, RNA was obtained, and then dissolved in DEPC water. Concentration and purity of RNA were detected by spectrophotometer (Thermo Scientific).

Operation steps for RNA reverse transcription: using PrimeScript™ II 1st strand cDNA Synthesis Kit (TaKaRa, 6210) and operating RNA reverse transcription completely in accordance with the kit instructions. RNA content was adjusted to 5 ng. The primer was Oligo dT Primer. Program Reverse transcription PCR was as follows: 42° C. for 50 min, 95° C. for 5 min, and 4° C. for preservation, and the PCR instrument was a SimpliAmp™ thermal cycle instrument (ABI).

Operating steps for fluorescence quantitative PCR: using Maxima SYBR Green qPCR Master Mix (Thermo Scientific, K0251) and a kit, and operating fluorescence quantitative PCR completely in accordance with the kit instruction. After adding the reaction solution as required, and placing the reaction plate in a qPCR instrument for detection. The reaction program was as follows: 95° C. for 10 min, [95° C. for 15 s, 60° C. for 30 s]× 40 cycles, 72° C. for 30 s, and 72° C. for 10 min. The expression of genes was obtained at different time points (FIG. 6B).

The primer sequences used for qPCR were as follows (5′-3′):

OCT4 primer sequences:
Forward:
GAAGCAGAAGAGGATCACCTTG
Reverse:
TTCTTAAGGCTGAGCTGCAAG
Nanog primer sequences:
Forward:
CCTCAGCCTCCAGCAGATGC
Reverse:
CCGCTTGCACTTCACCCTTTG

Although the present disclosure has been described in detail with general description and specific embodiments, some modifications or improvements can be made on the basis of the present disclosure, which are obvious for people skilled in the art. Therefore, these modifications or improvements made without deviating from the spirit of the present disclosure belong to the scope of protection required by the present disclosure.

### INDUSTRIAL APPLICABILITY

The present disclosure provides a scaffold with hierarchical structure and preparation method therefor and application thereof. The scaffold with hierarchical structure provided by the present disclosure has a structure from centimeter scale to micron scale, which can be used in fields of three-dimensional cell culture, in vitro large-scale amplification, in vitro tissue-like construction, tissue engineering and regenerative medicine, pathological model research, new drug development and drug toxicology research. The scaffold with hierarchical structure has the characteristics of customizable macro structure, adjustable hierarchical structure and pore size, high porosity, permeability, cell load and elastic modulus, good mechanical properties and cell functions and non-destructive collection of cells, and has good economic value and application prospect.

1. A scaffold with hierarchical structure, comprising a scaffold body, wherein,
there are big interconnected pores with an average pore diameter of 10 to 500 μm;
a porosity of the scaffold body is 10% to 95%; and
a Young's modulus of 0.1 kPa to 10 MPa.

2. The scaffold with hierarchical structure according to claim 1, wherein,
a macro structure of the scaffold body is columnar, blocky, lamellar, cystic or tubular; and/or
the scaffold body is a cylinder, a cube or a prism; and/or
there are big interconnected pores with an average pore diameter of 80 to 200 μm inside the scaffold body; and/or
the porosity of the scaffold body is 50% to 95%; and/or
the Young's modulus of the scaffold body is 30 to 500 kPa.

3. The scaffold with hierarchical structure according to claim 1 wherein,
the scaffold body further comprises at least one hollow channel; preferably, the hollow channel runs through the top and bottom of the scaffold body; further preferably, the at least one hollow channel is two, three, four or more hollow channels; and/or, further preferably, a diameter of the hollow channel is 0.1 to 5 cm;
and/or, a ratio of height to diameter of the scaffold body is (0.1 to 10) : (10 to 0.1), preferably 1:1;
and/or, the height of the scaffold body is 0.1 to 8 cm, preferably 1 cm; and/or, the diameter of the scaffold body is 0.1 to 8 cm, preferably 1 cm;
and/or, the porosity of the scaffold body is 75% to 95%.

4. The scaffold with hierarchical structure according to claim 1 wherein,
the scaffold body comprises a three-dimensional structure with an upper size of 0.5 to 50 cm; preferably, the dimension of the three-dimensional structure is 1 cm×1 cm×0.5 cm; and/or
the scaffold body is composed of a microfilament material of about 50 to 800 μm; and/or
the scaffold body comprises hollow channels with an interval of 0.1 to 1000 mm.

5. The scaffold with hierarchical structure according to claims 1, wherein, when the scaffold with hierarchical structure is compressed, the scaffold with hierarchical structure exhibits at least 20% to 70% or higher compression strain without permanent deformation or mechanical damage.

6. The scaffold with hierarchical structure according to claim 1, wherein, the scaffold body is made of a biocompatible material;
preferably, the biocompatible material is selected from a natural material and/or an artificial synthetic material;
further preferably, the natural material is at least one selected from alginate, alginate derivatives, gelatin, gelatin derivatives, agar, matrix gel, collagen, collagen derivatives, hyaluronic acid, hyaluronic acid derivatives, cellulose, cellulose derivatives, proteoglycan, proteoglycan derivatives, glycoprotein, glycoprotein derivatives, chitosan, chitosan derivatives, laminin, fibronectin, fibrin, silk fibroin, silk fibroin derivatives, vitronectin, osteopontin, peptide hydrogel and DNA hydrogel, and preferably the natural material is sodium alginate and/or gelatin; and/or
further preferably, the synthetic material is at least one selected from polyglycolic acid, polylactic acid, polylactic acid-glycolic acid copolymer, polyglutamic acid-polyethylene glycol, polycaprolactone, polytrimethylene carbonate, polyglycolic acid, polyethylene glycol-polydioxanone, polyethylene glycol, polytetrafluoroethylene, polyoxyethylene, polyethylene vinyl acetate, polytrimethylene carbonate, poly(p-dioxanone), polyether ether ketone, and derivatives and polymers thereof, and preferably the synthetic material is polyglycolic acid or polylactic acid, and/or
further preferably, the crosslinking agent used for preparing the scaffold body is at least one selected from divalent cation, genipin, glutaraldehyde, adopyl diacidhydrizine, epichlorohydrin, carbodiimide, thrombin and derivatives thereof, and preferably the crosslinking agent is calcium chloride; and
further preferably, the scaffold body is made of polyglycolic acid and fibrin, and the crosslinking agent is thrombin.

7. A preparation method for the scaffold with hierarchical structure according to claim 1, comprising the following steps:
1) preparing a precursor solution with a biocompatible material and a corresponding crosslinking agent;
2) preparing a three-dimensional structure body using the precursor solution as raw material;
3) freezing the three-dimensional structure body; and
4) drying the frozen three-dimensional structure body to obtaining the scaffold with hierarchical structure,
wherein, preferably, a mass percentage concentration of the biocompatible material is 0.1% to 80%, and more preferably 1% to 25%; and/or
preferably, a mass percentage concentration of the crosslinking solution is 0.1 mM to 10 M, and preferably 1 mM to 100 mM; and/or
preferably, the biocompatible material and the crosslinking agent solution are mixed according to a volume ratio of from 1000:1 to 1:1000, and preferably from 10:1 to 1:10; and/or
preferably, the precursor solution is made of a polyglycolic acid solution with a concentration of 1% to 25%, a fibrinogen solution with a concentration of 1% to 25% and a thrombin solution with a concentration of 1 to 2000 mM; and/or
preferably, the three-dimensional structure body is subjected to stepwise freezing and more preferably incubated at 4° C. for 0.5 to 24 h, then at −20° C. for 0.5 to 48 h, and then at −80° C. for 0.5 to 48 h; and/or
preferably, drying the frozen three-dimensional structure body by vacuum freeze drying, and more preferably under a condition of −4° C. to −80° C. and 1 to 1000 Pa.

8. The scaffold with hierarchical structure prepared by the preparation method according to claim 7.

9. (canceled)

10. A three-dimensional cell culture method, comprising: inoculating cells or a mixture of cells and a biocompatible material into the scaffold with hierarchical structure according to claim 1 for three-dimensional culture; or, further, comprising a step of cell collection and/or detection; wherein
preferably, the cells are selected from one or more of the following cells: embryonic stem cells from various sources, pluripotent stem cells, induced pluripotent stem cells, stem cells from various organs, progenitor cells from various organs, mesenchymal stem cells, cells differentiated from various stem cells by induced differentiation, fibroblasts from various organs, epithelial cells from various organs, epidermal cells from various organs, endothelial cells from various organs, muscle cells from various organs, amniotic cells, cone cells, nerve cells, blood cells, red blood cells, white blood cells, platelets, vascular cells, phagocytes, immune cells, lymphocytes, eosinophils, basophils, plasma cells, mast cells, antigen presenting cells, cells of mononuclear phagocyte system, melanocytes, chondrocytes, bone-derived cells, smooth muscle cells, skeletal muscle cells, cardiac muscle cells, secretory cells, adipocytes, ciliated cells, pancreatic cells, renal cells, intestinal mucosa cells, hepatocytes, stem cells or progenitor cells from liver, hepatic macrophages, kupffer cells, astrocytes, biliary epithelial cells, sinusoidal endothelial cells and cells from other tissues and organs, and various tumor cells, various cells for immunotherapy, various cells and cell lines obtained after gene editing, and virus packaging or modification; and further preferably, the cells are stem cells, and more preferably embryonic stem cells or liver stem cells;
and/or, preferably, the biocompatible material is at least one material of alginate, alginate derivatives, gelatin, gelatin derivatives, agar, matrix gel, collagen, collagen derivatives, hyaluronic acid, hyaluronic acid derivatives, cellulose, cellulose derivatives, proteoglycan, proteoglycan derivatives, glycoprotein, glycoprotein derivatives, chitosan, chitosan derivatives, laminin, fibronectin and fibrin, silk fibroin, silk fibroin derivatives, vitronectin, osteopontin, peptide hydrogel, and DNA hydrogel, and preferably the biocompatible material is collagen and derivatives thereof.

11. The scaffold with hierarchical structure according to claim 2, wherein,
the scaffold body further comprises at least one hollow channel; preferably, the hollow channel runs through the top and bottom of the scaffold body; further preferably, the at least one hollow channel is two, three, four or more hollow channels; and/or, further preferably, a diameter of the hollow channel is 0.1 to 5 cm;
and/or, a ratio of height to diameter of the scaffold body is (0.1 to 10) : (10 to 0.1), preferably 1:1;
and/or, the height of the scaffold body is 0.1 to 8 cm, preferably 1 cm; and/or, the diameter of the scaffold body is 0.1 to 8 cm, preferably 1 cm;
and/or, the porosity of the scaffold body is 75% to 95%.

12. The scaffold with hierarchical structure according to claim 2, wherein,
the scaffold body comprises a three-dimensional structure with an upper size of 0.5 to 50 cm; preferably, the dimension of the three-dimensional structure is 1 cm×1 cm×0.5 cm; and/or
the scaffold body is composed of a microfilament material of about 50 to 800 μm; and/or
the scaffold body comprises hollow channels with an interval of 0.1 to 1000 mm.

13. The scaffold with hierarchical structure according to claim 2, wherein, when the scaffold with hierarchical structure is compressed, the scaffold with hierarchical structure exhibits at least 20% to 70% or higher compression strain without permanent deformation or mechanical damage.

14. The scaffold with hierarchical structure according to claim 3, wherein, when the scaffold with hierarchical structure is compressed, the scaffold with hierarchical structure exhibits at least 20% to 70% or higher compression strain without permanent deformation or mechanical damage.

15. The scaffold with hierarchical structure according to claim 4, wherein, when the scaffold with hierarchical structure is compressed, the scaffold with hierarchical structure exhibits at least 20% to 70% or higher compression strain without permanent deformation or mechanical damage.

16. The scaffold with hierarchical structure according to claim 2, wherein, the scaffold body is made of a biocompatible material;
preferably, the biocompatible material is selected from a natural material and/or an artificial synthetic material;
further preferably, the natural material is at least one selected from alginate, alginate derivatives, gelatin, gelatin derivatives, agar, matrix gel, collagen, collagen derivatives, hyaluronic acid, hyaluronic acid derivatives, cellulose, cellulose derivatives, proteoglycan, proteoglycan derivatives, glycoprotein, glycoprotein derivatives, chitosan, chitosan derivatives, laminin, fibronectin, fibrin, silk fibroin, silk fibroin derivatives, vitronectin, osteopontin, peptide hydrogel and DNA hydrogel, and preferably the natural material is sodium alginate and/or gelatin; and/or
further preferably, the synthetic material is at least one selected from polyglycolic acid, polylactic acid, polylactic acid-glycolic acid copolymer, polyglutamic acid-polyethylene glycol, polycaprolactone, polytrimethylene carbonate, polyglycolic acid, polyethylene glycol-polydioxanone, polyethylene glycol, polytetrafluoroethylene, polyoxyethylene, polyethylene vinyl acetate, polytrimethylene carbonate, poly(p-dioxanone), polyether ether ketone, and derivatives and polymers thereof, and preferably the synthetic material is polyglycolic acid or polylactic acid, and/or
further preferably, the crosslinking agent used for preparing the scaffold body is at least one selected from divalent cation, genipin, glutaraldehyde, adopyl diacidhydrizine, epichlorohydrin, carbodiimide, thrombin and derivatives thereof, and preferably the crosslinking agent is calcium chloride; and
further preferably, the scaffold body is made of polyglycolic acid and fibrin, and the crosslinking agent is thrombin.

17. The scaffold with hierarchical structure according to claim 3, wherein, the scaffold body is made of a biocompatible material;
preferably, the biocompatible material is selected from a natural material and/or an artificial synthetic material;
further preferably, the natural material is at least one selected from alginate, alginate derivatives, gelatin, gelatin derivatives, agar, matrix gel, collagen, collagen derivatives, hyaluronic acid, hyaluronic acid derivatives, cellulose, cellulose derivatives, proteoglycan, proteoglycan derivatives, glycoprotein, glycoprotein derivatives, chitosan, chitosan derivatives, laminin, fibronectin, fibrin, silk fibroin, silk fibroin derivatives, vitronectin, osteopontin, peptide hydrogel and DNA hydrogel, and preferably the natural material is sodium alginate and/or gelatin; and/or
further preferably, the synthetic material is at least one selected from polyglycolic acid, polylactic acid, polylactic acid-glycolic acid copolymer, polyglutamic acid-polyethylene glycol, polycaprolactone, polytrimethylene carbonate, polyglycolic acid, polyethylene glycol-polydioxanone, polyethylene glycol, polytetrafluoroethylene, polyoxyethylene, polyethylene vinyl acetate, polytrimethylene carbonate, poly(p-dioxanone), polyether ether ketone, and derivatives and polymers thereof, and preferably the synthetic material is polyglycolic acid or polylactic acid, and/or
further preferably, the crosslinking agent used for preparing the scaffold body is at least one selected from divalent cation, genipin, glutaraldehyde, adopyl diacidhydrizine, epichlorohydrin, carbodiimide, thrombin and derivatives thereof, and preferably the crosslinking agent is calcium chloride; and
further preferably, the scaffold body is made of polyglycolic acid and fibrin, and the crosslinking agent is thrombin.

18. The scaffold with hierarchical structure according to claim 4, wherein, the scaffold body is made of a biocompatible material;
preferably, the biocompatible material is selected from a natural material and/or an artificial synthetic material;
further preferably, the natural material is at least one selected from alginate, alginate derivatives, gelatin, gelatin derivatives, agar, matrix gel, collagen, collagen derivatives, hyaluronic acid, hyaluronic acid derivatives, cellulose, cellulose derivatives, proteoglycan, proteoglycan derivatives, glycoprotein, glycoprotein derivatives, chitosan, chitosan derivatives, laminin, fibronectin, fibrin, silk fibroin, silk fibroin derivatives, vitronectin, osteopontin, peptide hydrogel and DNA hydrogel, and preferably the natural material is sodium alginate and/or gelatin; and/or
further preferably, the synthetic material is at least one selected from polyglycolic acid, polylactic acid, polylactic acid-glycolic acid copolymer, polyglutamic acid-polyethylene glycol, polycaprolactone, polytrimethylene carbonate, polyglycolic acid, polyethylene glycol-polydioxanone, polyethylene glycol, polytetrafluoroethylene, polyoxyethylene, polyethylene vinyl acetate, polytrimethylene carbonate, poly(p-dioxanone), polyether ether ketone, and derivatives and polymers thereof, and preferably the synthetic material is polyglycolic acid or polylactic acid, and/or
further preferably, the crosslinking agent used for preparing the scaffold body is at least one selected from divalent cation, genipin, glutaraldehyde, adopyl diacidhydrizine, epichlorohydrin, carbodiimide, thrombin and derivatives thereof, and preferably the crosslinking agent is calcium chloride; and
further preferably, the scaffold body is made of polyglycolic acid and fibrin, and the crosslinking agent is thrombin.

19. The scaffold with hierarchical structure according to claim 5, wherein, the scaffold body is made of a biocompatible material;
preferably, the biocompatible material is selected from a natural material and/or an artificial synthetic material;
further preferably, the natural material is at least one selected from alginate, alginate derivatives, gelatin, gelatin derivatives, agar, matrix gel, collagen, collagen derivatives, hyaluronic acid, hyaluronic acid derivatives, cellulose, cellulose derivatives, proteoglycan, proteoglycan derivatives, glycoprotein, glycoprotein derivatives, chitosan, chitosan derivatives, laminin, fibronectin, fibrin, silk fibroin, silk fibroin derivatives, vitronectin, osteopontin, peptide hydrogel and DNA hydrogel, and preferably the natural material is sodium alginate and/or gelatin; and/or
further preferably, the synthetic material is at least one selected from polyglycolic acid, polylactic acid, polylactic acid-glycolic acid copolymer, polyglutamic acid-polyethylene glycol, polycaprolactone, polytrimethylene carbonate, polyglycolic acid, polyethylene glycol-polydioxanone, polyethylene glycol, polytetrafluoroethylene, polyoxyethylene, polyethylene vinyl acetate, polytrimethylene carbonate, poly(p-dioxanone), polyether ether ketone, and derivatives and polymers thereof, and preferably the synthetic material is polyglycolic acid or polylactic acid, and/or
further preferably, the crosslinking agent used for preparing the scaffold body is at least one selected from divalent cation, genipin, glutaraldehyde, adopyl diacidhydrizine, epichlorohydrin, carbodiimide, thrombin and derivatives thereof, and preferably the crosslinking agent is calcium chloride; and
further preferably, the scaffold body is made of polyglycolic acid and fibrin, and the crosslinking agent is thrombin.
